SEQUENCE LISTING (55 sequences)

SEQ ID NO: 1 - protein, 5 aa, synthetic construct
  REGION 1..5: note = antibody sequence
SYAMG

SEQ ID NO: 2 - protein, 17 aa, synthetic construct
  REGION 1..17: note = antibody sequence
AISWSGGTTYYADSVKG

SEQ ID NO: 3 - protein, 16 aa, synthetic construct
  REGION 1..16: note = antibody sequence
SLDCSGPGCHTAEYDY

SEQ ID NO: 4 - protein, 5 aa, synthetic construct
  REGION 1..5: note = antibody sequence
NYGMG

SEQ ID NO: 5 - protein, 17 aa, synthetic construct
  REGION 1..17: note = antibody sequence
GISWSGGSTDYADSVKG

SEQ ID NO: 6 - protein, 17 aa, synthetic construct
  REGION 1..17: note = antibody sequence
VFSGAETAYYPSDDYDY

SEQ ID NO: 7 - protein, 5 aa, synthetic construct
  REGION 1..5: note = antibody sequence
NYGMG

SEQ ID NO: 8 - protein, 17 aa, synthetic construct
  REGION 1..17: note = antibody sequence
GITWSGGSTHYADLVKG

SEQ ID NO: 9 - protein, 17 aa, synthetic construct
  REGION 1..17: note = antibody sequence
VFSGAETAYYPSTEYDY

SEQ ID NO: 10 - protein, 5 aa, synthetic construct
  REGION 1..5: note = antibody sequence
NYGMG

SEQ ID NO: 11 - protein, 17 aa, synthetic construct
  REGION 1..17: note = antibody sequence
GISWSGGSTYYADSVKG

SEQ ID NO: 12 - protein, 17 aa, synthetic construct
  REGION 1..17: note = antibody sequence
VFSGAETAQYPSYDYDY

SEQ ID NO: 13 - protein, 5 aa, synthetic construct
  REGION 1..5: note = antibody sequence
  VARIANT 5: note = X is G or S
NYAMX

SEQ ID NO: 14 - protein, 17 aa, synthetic construct
  REGION 1..17: note = antibody sequence
AISWSGGSTSYADSVKG

SEQ ID NO: 15 - protein, 21 aa, synthetic construct
  REGION 1..21: note = antibody sequence
  VARIANT 7: note = X is Y, F or S
QFSGADXGFGRLGIRGYEYDY

SEQ ID NO: 16 - protein, 5 aa, synthetic construct
  REGION 1..5: note = antibody sequence
NYAMG

SEQ ID NO: 17 - protein, 17 aa, synthetic construct
  REGION 1..17: note = antibody sequence
AISWSGGSTYYADSVKG

SEQ ID NO: 18 - protein, 21 aa, synthetic construct
  REGION 1..21: note = antibody sequence
MFSGSESQLVVVITNLYEYDY

SEQ ID NO: 19 - protein, 5 aa, synthetic construct
  REGION 1..5: note = antibody sequence
NYAMG

SEQ ID NO: 20 - protein, 17 aa, synthetic construct
  REGION 1..17: note = antibody sequence
TISWSGGSTYYADSVKG

SEQ ID NO: 21 - protein, 18 aa, synthetic construct
  REGION 1..18: note = antibody sequence
AFSGSDYANTKKEVEYDY

SEQ ID NO: 22 - protein, 5 aa, synthetic construct
  REGION 1..5: note = antibody sequence
DYCIA

SEQ ID NO: 23 - protein, 17 aa, synthetic construct
  REGION 1..17: note = antibody sequence
CITTSDGSTYYADSVKG

SEQ ID NO: 24 - protein, 18 aa, synthetic construct
  REGION 1..18: note = antibody sequence
YFGYGCYGGAQDYRAMDY

SEQ ID NO: 25 - protein, 127 aa, synthetic construct
  REGION 1..127: note = antibody sequence
EVQLVESGGGLVQAGGSLRLSCAASGRPFSNYAMGWFRQAPGKEREFVTVISWSGGSTYYADSVKGRFTISRDNAKNTVYLQMNSLKPEDTAVYYCAAQFSGASTVVAGTALDYDYWGQGTRVTVSS

SEQ ID NO: 26 - protein, 126 aa, synthetic construct
  REGION 1..126: note = antibody sequence
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSS

SEQ ID NO: 27 - protein, 126 aa, synthetic construct
  REGION 1..126: note = antibody sequence
EVQLVESGGGLVQAGGSLRLSCAASGRPFSNYGMGWFRQAPGKKREFVAGISWSGGSTDYADSVKGRLTISRDNAKNTVYLQMNSLKPEDTAVYYCAAVFSGAETAYYPSDDYDYWGQGTQVTVSS

SEQ ID NO: 28 - protein, 126 aa, synthetic construct
  REGION 1..126: note = antibody sequence
EVQLVESGGGLVQAGGSLRLSCAASGRPFSNYGMGWFRQAPGKKRESVAGISWSGGSTDYADSVKGRFTISRDNAKNTVYLQMNSLKPEDTAVYYCAAVFSGAETAYYPSDDYDYWGQGTQVTVSS

SEQ ID NO: 29 - protein, 126 aa, synthetic construct
  REGION 1..126: note = antibody sequence
EVQLVESGGGLVQAGGSLRLSCAVSGRPFSNYGMGWFRQAPGKEREFVAGITWSGGSTHYADLVKGRFTISRDNAKNTVHLQMNSLKPEDTAVYYCAAVFSGAETAYYPSTEYDYWGQGTQVTVSS

SEQ ID NO: 30 - protein, 126 aa, synthetic construct
  REGION 1..126: note = antibody sequence
EVQLVESGGGLVQAGGSLRLSCAASGRPFNNYGMGWFRQAPGKEREFVAGISWSGGSTYYADSVKGRFTISRDNAKNTVYLQMNSLKPEDTAVYYCAAVFSGAETAQYPSYDYDYWGQGTQVTVSS

SEQ ID NO: 31 - protein, 130 aa, synthetic construct
  REGION 1..130: note = antibody sequence
  VARIANT 35: note = X is G or S
  VARIANT 105: note = X is Y, F or S
EVQLVESGGGLVQAGGSLRLSCAASGRPFSNYAMXWFRQAPGKEREFVAAISWSGGSTSYADSVKGRFTISRDNAKNTVYLQMNSPKPEDTAIYYCAAQFSGADXGFGRLGIRGYEYDYWGQGTQVTVSS

SEQ ID NO: 32 - protein, 130 aa, synthetic construct
  REGION 1..130: note = antibody sequence
EVQLVESGGGLVQAGGSLRLSCAASGRTFSNYAMGWFRQAPGKEREFVAAISWSGGSTYYADSVKGRFTISRDNAKNTVYLQMNSLKPEDTAVYYCAAMFSGSESQLVVVITNLYEYDYWGQGTQVTVSS

SEQ ID NO: 33 - protein, 127 aa, synthetic construct
  REGION 1..127: note = antibody sequence
EVQLVESGGGLVQAGGSLRLSCAASGRTFSNYAMGWFRQAPGKEREFVATISWSGGSTYYADSVKGRFTISRDNAKNTVYLQMNSLKPEDTAVYYCAAAFSGSDYANTKKEVEYDYWGQGTQVTVSS

SEQ ID NO: 34 - protein, 127 aa, synthetic construct
  REGION 1..127: note = antibody sequence
EVQLVESGGGLVQAGGSLRLSCAASGFTFDDYCIAWFRQAPGKEREPVSCITTSDGSTYYADSVKGRFTISSDNAKNTVYLQMNRLKPEDTAVYYCAAYFGYGCYGGAQDYRAMDYWGKGTLVTVSS

SEQ ID NO: 35 - protein, 5 aa, synthetic construct
  REGION 1..5: note = antibody sequence
DNVMG

SEQ ID NO: 36 - protein, 16 aa, synthetic construct
  REGION 1..16: note = antibody sequence
TIRTGGSTNYADSVKG

SEQ ID NO: 37 - protein, 11 aa, synthetic construct
  REGION 1..11: note = antibody sequence
TIPVPSTPYDY

SEQ ID NO: 38 - protein, 119 aa, synthetic construct
  REGION 1..119: note = antibody sequence
QVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSS

SEQ ID NO: 39 - protein, 254 aa, synthetic construct
  REGION 1..254: note = antibody sequence
EVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSSGGGGSEVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTQVTVSS

SEQ ID NO: 40 - protein, 5 aa, synthetic construct
  REGION 1..5: note = antibody sequence
SYGMG

SEQ ID NO: 41 - protein, 17 aa, synthetic construct
  REGION 1..17: note = antibody sequence
GISWRGDSTGYADSVKG

SEQ ID NO: 42 - protein, 15 aa, synthetic construct
  REGION 1..15: note = antibody sequence
AAGSAWYGTLYEYDY

SEQ ID NO: 43 - protein, 124 aa, synthetic construct
  REGION 1..124: note = antibody sequence
EVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 44 - protein, 5 aa, synthetic construct
  REGION 1..5: note = antibody sequence
RSAMG

SEQ ID NO: 45 - protein, 17 aa, synthetic construct
  REGION 1..17: note = antibody sequence
AIGTRGGSTKYADSVKG

SEQ ID NO: 46 - protein, 17 aa, synthetic construct
  REGION 1..17: note = antibody sequence
RGPGYPSAAIFQDEYHY

SEQ ID NO: 47 - protein, 126 aa, synthetic construct
  REGION 1..126: note = antibody sequence
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSS

SEQ ID NO: 48 - protein, 10 aa, synthetic construct
  REGION 1..10: note = antibody sequence
RFMISEYSMH

SEQ ID NO: 49 - protein, 16 aa, synthetic construct
  REGION 1..16: note = antibody sequence
TINPAGTTDYADSVKG

SEQ ID NO: 50 - protein, 5 aa, synthetic construct
  REGION 1..5: note = antibody sequence
DGYGY

SEQ ID NO: 51 - protein, 111 aa, synthetic construct
  REGION 1..111: note = antibody sequence
EVQLVESGGGLVQPGGSLRLSCAASRFMISEYSMHWVRQAPGKGLEWVSTINPAGTTDYADSVKGRFTISRDNAKNTLYLQMNSLRAEDTAVYYCDGYGYRGQGTQVTVSS

SEQ ID NO: 52 - protein, 10 aa, synthetic construct
  REGION 1..10: note = antibody sequence
  VARIANT 10: note = X is G or S
GRTASSYVMX

SEQ ID NO: 53 - protein, 17 aa, synthetic construct
  REGION 1..17: note = antibody sequence
  VARIANT 12: note = X is A or T
VINWNGDSTYYXDSVKG

SEQ ID NO: 54 - protein, 22 aa, synthetic construct
  REGION 1..22: note = antibody sequence
  VARIANT 11: note = X is Y or F
DTRREWYRDGXWGPPARYEYDY

SEQ ID NO: 55 - protein, 131 aa, synthetic construct
  REGION 1..131: note = antibody sequence
EVQLVESGGGLVQAGGSLRLSCAASGRTASSYVMGWFRQAPGKEREFVAVINWNGDSTYYTDSVKGRFAISRDNAKNTVYLQMNSLKPEDTAVYYCAADTRREWYRDGYWGPPARYEYDYRGQGTQVTVSS

(19) United States
(12) Patent Application Publication
(10) Pub. No.: US 2025/0099581 A1
(43) Pub. Date: Mar. 27, 2025

(54) USES OF GAMMA DELTA T CELL ACTIVATING ANTIBODIES

(71) Applicant: LAVA THERAPEUTICS N.V., Utrecht (NL)

(72) Inventors: Johannes Jelle VAN DER VLIET, Amsterdam (NL); Paul Willem Henri Ida PARREN, Utrecht (NL); Jurjen Matthijs RUBEN, Utrecht (NL); Erik ENSING, Utrecht (NL)

(21) Appl. No.: 18/640,858

(22) Filed: Apr. 19, 2024

Related U.S. Application Data
(63) Continuation of application No. PCT/EP2022/079374, filed on Oct. 21, 2022.
(60) Provisional application No. 63/274,711, filed on Nov. 2, 2021.
(60) Provisional application No. 63/270,267, filed on Oct. 21, 2021.

(30) Foreign Application Priority Data
Oct. 21, 2021 (EP) 21204014.1

(51) Int. Cl. A61K 39/395; A61K 31/663; A61K 31/675; A61K 39/00; A61P 35/00

(52) U.S. Cl. CPC A61K 39/39558; A61K 31/663; A61K 31/675; A61P 35/00; A61K 2039/505; A61K 2039/545

(57) ABSTRACT

The present invention relates to novel uses of gamma delta T-cell activating antibodies, in particular uses of such antibodies in patients that are also being treated, or have recently been treated, with aminobisphosphonates, or other inhibitors of the mevalonate pathway that inhibit farnesyl pyrophosphate synthase or an enzyme further downstream in the mevalonate pathway.

### CROSS-REFERENCE TO RELATED APPLICATIONS

This application is a continuation of International Application No. PCT/EP2022/079374, filed Oct. 21, 2022, which claims priority to EP Application Serial No. 21204014.1, filed Oct. 21, 2021; U.S. Provisional Application No. 63/270,267, filed Oct. 21, 2021; and U.S. Provisional Application No. 63/274,711, filed Nov. 2, 2021, the disclosures of each are hereby incorporated by reference in their entireties.

### REFERENCE TO AN ELECTRONIC SEQUENCE LISTING

The contents of the electronic sequence listing (LVAT_007_02US_SubSeqList_ST26.xml; Size: 74,085 bytes; and Date of Creation: Apr. 25, 2024) are herein incorporated by reference in its entirety.

### FIELD OF THE INVENTION

The present invention relates to novel uses of gamma delta (γδ) T cell activating antibodies, in particular uses of such antibodies in patients that are also being treated, or have recently been treated, with aminobisphosphonates, or other inhibitors of the mevalonate pathway that inhibit farnesyl pyrophosphate synthase (FPPS) or an enzyme further downstream in the mevalonate pathway.

### BACKGROUND OF THE INVENTION

Aminobisphosphonates (NBPs), such as zoledronate and alendronate, are compounds frequently used in the treatment or prevention of osteoporosis, hypercalcemia and osteolytic destruction by metastases of solid tumors (e.g. breast and prostate cancer) or hematological neoplasias (e.g. multiple myeloma).

NBPs selectively interfere with the enzymatic activity of FPPS. NBPs (as they target FPPS downstream to HMGCoAR) increase intracellular levels of isopentenyl pyrophosphate (IPP) and stimulate its release in the extracellular space. Interestingly, IPP is very similar to natural ligands of the Vγ9Vδ2 T-cell receptor (TCR), which is expressed by a unique subset of unconventional T cells deeply involved in innate immune responses against microbes as well as stressed and transformed cells.

NBPs have been used in γδ T-cell-based immunotherapy to activate and expand this subset of T cells, in particular Vγ9Vδ2 T cells, in patients.

A further potential cancer treatment involving γδ T cells is the use of γδ T cell engagers (γδ TCEs, i.e. multispecific antibodies that bind the γδ T cell receptor and a tumor cell target) to mediate killing of tumor cells by γδ T cells.

It has been observed that repetitive treatment with NBPs leads to exhaustion, anergy and depletion of γδ T cells (Sicard et al. (2005) J Immunol 175:5471, also discussed in Oberg et al. (2014) Front Immunol 2014, 5:1). Therefore, until the present invention, it was thought that γδ T cells would be unresponsive to γδ TCEs following repetitive treatment with NBPs.

### SUMMARY OF THE INVENTION

It has now surprisingly been found that Vγ9Vδ2 T cell-containing peripheral blood mononuclear cells (PBMCs) of patients that have received repetitive treatment with an NBP are still responsive to γδ TCEs and that γδ TCEs can mediate killing of tumor cells by PBMCs of such patients. Furthermore, surprisingly, γδ T cells of patients that had received repetitive treatment with an NBP were both able to kill tumor cells in the presence of γδ TCEs and to expand in the presence of γδ TCEs.

This allows for cancer treatment regimens wherein γδ TCEs are administered to patients that simultaneously also receive, or have received, repetitive NBP treatment. In particular, γδ TCE treatment is anticipated also to be beneficial for cancer patients that are treated with NBPs to avoid hypercalcemia or osteolytic destruction or for patients that are similarly treated for osteoporosis or prevention thereof.

In a first aspect, the invention relates to a multispecific antibody comprising a first antigen-binding region that binds a human Vγ9Vδ2 T cell receptor and a second antigen-binding region that binds a target expressed on tumor cells for use in the treatment of cancer in a human subject, wherein said human subject is also being treated with, or has also been treated with an aminobisphosphonate (NBP) or other inhibitor of the mevalonate pathway, wherein the inhibitor inhibits farnesyl pyrophosphate synthase or an enzyme further downstream in said pathway,
and wherein the first, or a subsequent, administration of said multispecific antibody is performed after the human subject has received two or more administrations of said NBP or other inhibitor.

### DESCRIPTION OF THE FIGURES

FIG. 1: Panel A: Vγ9Vδ2 T cells degranulation was assessed after overnight coculture of PBMCs with MM1s.CD1d target cells, in the presence of the targeting γδ TCE or medium control (−). CD107a expression on Vγ9Vδ2 T cells was assessed by flow cytometry as a measure for degranulation (n=8). Panel B shows the same data, but links the data with and without γδ TCE for individual patients. Closed circles represent zoledronate treated patients (n=4), open squares represent pamidronate treated patients (n=3) and triangles represent the clodronic acid treated patient (n=1).

FIG. 2: Relative lysis of MM1s.CD1d, following a 7-day coculture with Vγ9Vδ2 T cells in the presence of a γδ TCE or medium control (−).

FIG. 3: Vγ9Vδ2 T cell expansion was assessed relative to day 0, by culturing PBMC and MM1s.CD1d target cells in the presence of a γδ TCE or medium control (−) for 7 and 14 days.

FIG. 4: Vγ9Vδ2 T cell expansion was assessed relative to day 0, by culturing PBMC and MM1s.CD1d target cells in the presence of IL-2 and a γδ TCE or medium control (−) for 7 and 14 days.

### DETAILED DESCRIPTION OF THE INVENTION

### Definitions

The term “aminobisphosphonate” (abbreviated as NBP), when used herein, refers to a nitrogen-containing analogue of pyrophosphate that contains a carbon instead of an oxygen atom. NBPs have the following generic structure, wherein at least one of R1 and R2 contains a nitrogen:

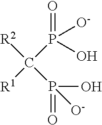

The term “human Vγ9Vδ2 T cell receptor” when used herein, refers to the Vγ9Vδ2 T cell receptor found on human γδ T cells.

The term “γδ T cell engager” (abbreviated as γδ TCE) refers to a molecule that has a binding specificity for a target on a γδ T cell and a further binding specificity for a target on a tumor cell. By binding to both these targets, γδ TCEs recruit T cells to a tumor cell or tumor environment.

The term “human Vδ2”, when used herein, refers to the rearranged 02 chain of the Vγ9Vδ2-T cell receptor (TCR). GenBank: CAA51166.1, gives an example of a δ2 sequence. TRDV2, T cell receptor delta variable 2, represents the variable region (UniProtKB-A0JD36 (AOJD36_HUMAN) gives an example of a TRDV2 sequence). “binding the Vδ2 chain of a Vγ9Vδ2-TCR” means that the antibody can bind the Vδ2 chain as a separate molecule and/or as part of a Vγ9Vδ2-TCR (T cell Receptor). However, the antibody will not bind to the Vγ9 chain as a separate molecule.

The term “human Vγ9”, when used herein, refers to the rearranged γ9 chain of the Vγ9Vδ2-T cell receptor (TCR). GenBank: NG_001336.2 gives an example of a γ9 sequence. TRGV9, T cell receptor gamma variable 9 represents the variable region (UniProtKB-Q99603_HUMAN gives an example of a TRGV9 sequence).

The term “immunoglobulin” as used herein is intended to refer to a class of structurally related glycoproteins typically consisting of two pairs of polypeptide chains, one pair of light (L) chains and one pair of heavy (H) chains, all four potentially inter-connected by disulfide bonds, although some mammalian species do not produce light chains and only make heavy-chain antibodies. The term “immunoglobulin heavy chain”, “heavy chain of an immunoglobulin” or “heavy chain” as used herein is intended to refer to one of the chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The heavy chain constant region further comprises a hinge region. Within the structure of the immunoglobulin (e.g. IgG), the two heavy chains are inter-connected via disulfide bonds in the hinge region. Equally to the heavy chains, each light chain is typically comprised of several regions; a light chain variable region (VL) and a light chain constant region (CL). Furthermore, the VH and VL regions may be subdivided into regions of hypervariability (or hypervariable regions which may be hypervariable in sequence and/or form structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences may be determined by use of various methods, e.g. the methods provided by Choitia and Lesk (1987) J. Mol. Biol. 196:901 or Kabat et al. (1991) Sequence of protein of immunological interest, fifth edition. NIH publication. Various methods for CDR determination and amino acid numbering can be compared on www.abysis.org (UCL).

The term “antibody” is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about one hour, at least about two hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). Antigen-binding regions which interact with an antigen may comprise variable regions of both the heavy and light chains of an immunoglobulin molecule or may comprise or consist of single-domain antigen-binding regions, for example a heavy chain variable region only. The “Fc region” of an immunoglobulin is defined as the fragment of an antibody which would be typically generated after digestion of an antibody with papain which includes the two CH2-CH3 regions of an immunoglobulin and a connecting region, e.g. a hinge region. The constant domain of an antibody heavy chain defines the antibody isotype, e.g. IgG1, IgG2, IgG3, IgG4, IgA1, IgA2, IgM, IgD, or IgE. The Fc-region mediates the effector functions of antibodies with cell surface receptors called Fc receptors and proteins of the complement system.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

Reference to amino acid positions in the Fc region/Fc domain in the present invention is according to the EU-numbering (Edelman et al., Proc Natl Acad Sci USA. 1969 May;63 (1): 78-85; Kabat et al., Sequences of proteins of immunological interest. 5th Edition-1991 NIH Publication No. 91-3242).

The term “isotype” as used herein, refers to the immunoglobulin (sub) class (for instance IgG1, IgG2, IgG3, IgG4, IgD, IgA, IgE, or IgM) or any allotype thereof, such as IgG1m(za) and IgG1m(f) that is encoded by heavy chain constant region genes. Each heavy chain isotype can be combined with either a kappa (κ) or lambda (λ) light chain. An antibody used in the invention can possess any isotype.

As indicated above, the term antibody as used herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, i.e. a monovalent fragment consisting of the VL, VH, CL and CH1 domains, or a monovalent antibody as described in WO2007059782; (ii) F(ab′) 2 fragments, i.e. bivalent fragments comprising two Fab fragments linked by a disulfide bridge at the hinge region; (iii) a Fd fragment consisting essentially of the VH and CH1 domains; and (iv) a Fv fragment consisting essentially of the VL and VH domains of a single arm of an antibody. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single-chain antibodies are encompassed within the term antibody unless otherwise indicated by context. The term antibody, unless specified otherwise, also includes polyclonal antibodies, monoclonal antibodies (mAbs), chimeric antibodies and humanized antibodies, and antibody fragments provided by any known technique, such as enzymatic cleavage, peptide synthesis, and recombinant techniques.

In some embodiments of the antibodies used in the invention, the first antigen-binding region or the second antigen-binding region, or both, is a single-domain antibody. Single-domain antibodies (sdAb, also called Nanobody®, or VHH) are well known to the skilled person, see e.g. Hamers-Casterman et al. (1993) Nature 363:446, Roovers et al. (2007) Curr Opin Mol Ther 9:327 and Krah et al. (2016) Immunopharmacol Immunotoxicol 38:21. Single-domain antibodies comprise a single CDR1, a single CDR2 and a single CDR3. Examples of single-domain antibodies are variable fragments of heavy-chain-only antibodies, antibodies that naturally do not comprise light chains, single-domain antibodies derived from conventional antibodies, and engineered antibodies. Single-domain antibodies may be derived from any species including mouse, human, camel, llama, shark, goat, rabbit, and cow. For example, naturally occurring VHH molecules can be derived from antibodies raised in Camelidae species, for example in camel, dromedary, llama, alpaca and guanaco. Like a whole antibody, a single-domain antibody is able to bind selectively to a specific antigen. Single-domain antibodies may contain only the variable domain of an immunoglobulin chain, i.e. CDR1, CDR2 and CDR3 and framework regions.

The term “parent antibody”, is to be understood as an antibody which is identical to an antibody used in the invention, but wherein the parent antibody does not have one or more of the specified mutations. A “variant” or “antibody variant” or a “variant of a parent antibody” used in the present invention is an antibody molecule which comprises one or more mutations as compared to a “parent antibody”. Amino acid substitutions may exchange a native amino acid for another naturally-occurring amino acid, or for a non-naturally-occurring amino acid derivative. The amino acid substitution may be conservative or non-conservative. In the context of the present invention, conservative substitutions may be defined by substitutions within the classes of amino acids reflected in one or more of the following three tables:

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), and |
| | Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, |
| | R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, |
| | Q, R, S, P, and T |
| Flexible residues | Q, T, K, S, G, N, D, E, and R |

In the context of the present invention, a substitution in a variant is indicated as:
Original amino acid-position-substituted amino acid;

The three-letter code, or one letter code, are used, including the codes Xaa and X to indicate amino acid residue. Accordingly, the notation “T366W” means that the variant comprises a substitution of threonine with tryptophan in the variant amino acid position corresponding to the amino acid in position 366 in the parent antibody.

Furthermore, the term “a substitution” embraces a substitution into any one of the other nineteen natural amino acids, or into other amino acids, such as non-natural amino acids. For example, a substitution of amino acid T in position 366 includes each of the following substitutions: 366A, 366C, 366D, 366G, 366H, 366F, 366I, 366K, 366L, 366M, 366N, 366P, 366Q, 366R, 366S, 366E, 366V, 366W, and 366Y.

“% sequence identity”, when used herein, refers to the number of identical nucleotide or amino acid positions shared by different sequences (i.e., % identity= # of identical positions/total # of positions×100), taking into account the number of gaps, and the length of each gap, which need to be introduced for optimal alignment. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4.

The term “full-length antibody” when used herein, refers to an antibody which contains all heavy and light chain constant and variable domains corresponding to those that are normally found in a wild-type antibody of that isotype.

The term “chimeric antibody” refers to an antibody wherein the variable region is derived from a non-human species (e.g. derived from rodents) and the constant region is derived from a different species, such as human. Chimeric antibodies may be generated by genetic engineering. Chimeric monoclonal antibodies for therapeutic applications are developed to reduce antibody immunogenicity.

The term “humanized antibody” refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanization of non-human therapeutic antibodies is performed to minimize its immunogenicity in man while such humanized antibodies at the same time maintain the specificity and binding affinity of the antibody of non-human origin.

The term “multispecific antibody” refers to an antibody having specificities for at least two different, such as at least three, typically non-overlapping, epitopes, due to the presence of two or more antigen-binding regions. Such epitopes may be on the same or on different target antigens. If the epitopes are on different targets, such targets may be on the same cell or different cells or cell types.

The term “bispecific antibody” refers to an antibody having specificities for two different, typically non-overlapping, epitopes, due to the presence of two antigen-binding regions. Such epitopes may be on the same or different targets. If the epitopes are on different targets, such targets may be on the same cell or different cells or cell types.

Examples of different classes of bispecific antibodies include, but are not limited to, (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to an extra Fab fragment or parts of a Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) scFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include, but are not limited to, the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287 (52): 43331-9, doi: 10.1074/jbc.M112.397869. Epub 2012 Nov. 1), DIG-body and PIG-body (Pharmabcine, WO2010134666, WO2014081202), the Strand Exchange Engineered Domain body (SEEDbody) (EMD Serono), the Biclonics (Merus, WO2013157953), FcAAdp (Regeneron), bispecific IgG1 and IgG2 (Pfizer/Rinat), Azymetric scaffold (Zymeworks/Merck,), mAb-Fv (Xencor), bivalent bispecific antibodies (Roche, WO2009080254) and DuoBody® molecules (Genmab).

Examples of recombinant IgG-like dual targeting molecules include, but are not limited, to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 March-April;5 (2): 208-18), approaches with common light chain, κλBodies (NovImmune, WO2012023053) and CovX-body® (CovX/Pfizer, Doppalapudi, V. R., et al 2007. Bioorg. Med. Chem. Lett. 17,501-506).

Examples of IgG fusion molecules include, but are not limited to, Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 February; 32 (2): 191-8), Ts2Ab (MedImmune/AZ, Dimasi et al.] Mol Biol. 2009 Oct. 30;393 (3): 672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include, but are not limited to, scFv/Fc Fusions (Academic Institution, Pearce et al Biochem Mol Biol Int. 1997 September;42 (6): 1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual (ScFv) 2-Fab (National Research Center for Antibody Medicine-China).

Examples of Fab fusion bispecific antibodies include, but are not limited to, F(ab)2 (Medarex/AMGEN), Dual-Action or Bis-Fab (Genentech), Dock-and-Lock® (DNL) (ImmunoMedics), Bivalent Bispecific (Biotecnoly and Fab-Fv (UCB-Celltech).

Examples of scFv-, diabody-based and domain antibodies include, but are not limited to, Bispecific T Cell Engager (BITE®) (Micromet, Tandem Diabody (Tandab) (Affimed), Dual Affinity Retargeting Technology (DART™) (MacroGenics), Single-chain Diabody (Academic, Lawrence FEBS Lett. 1998 Apr. 3;425 (3): 479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August;88 (6): 667-75), dual targeting nanobodies® (Ablynx, Hmila et al., FASEB J. 2010), dual targeting heavy chain only domain antibodies.

In some embodiments, the multispecific antibody used in the invention is in a VHH-Fc format, i.e. the antibody comprises two or more single-domain antigen-binding regions that are linked to each other via a human Fc region dimer. In this format, each single-domain antigen-binding region is fused to an Fc region polypeptide and the two fusion polypeptides form a dimeric bispecific antibody via disulfide bridges in the hinge region. Such constructs typically do not contain full, or any, CH1 or light chain sequences. FIG. 12B of WO06064136 provides an illustration of an example of this embodiment.

In the context of antibody binding to an antigen, the terms “binds” or “specifically binds” refer to the binding of an antibody to a predetermined antigen or target (e.g. human Vδ2) to which binding typically is with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less, e.g. when determined using flow cytometry as described in the Examples herein. Alternatively, apparent K_{D} values can be determined using for instance surface plasmon resonance (SPR) technology in a BIAcore 3000 instrument using the antigen as the ligand and the binding moiety or binding molecule as the analyte. Specific binding means that the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen. The degree with which the affinity is lower is dependent on the K_{D} of the binding moiety or binding molecule, so that when the K_{D} of the binding moiety or binding molecule is very low (that is, the binding moiety or binding molecule is highly specific), then the degree with which the affinity for the antigen is lower than the affinity for a non-specific antigen may be at least 10,000-fold. The term “K_{D}” (M), as used herein, refers to the dissociation equilibrium constant of a particular interaction between the antigen and the binding moiety or binding molecule.

“Capable of binding the Vδ2 chain of a Vγ9Vδ2-TCR” or “binding the Vδ2 chain of a Vγ9Vδ2-TCR” or the like means that the antibody can bind the Vδ2 chain as a separate molecule and/or as part of a Vγ9Vδ2-TCR. However, the antibody will not bind to the Vγ9 chain as a separate molecule.

The terms “first” and “second” antigen-binding regions when used herein do not refer to their orientation/position in the antibody, i.e. they have no meaning with regard to the N- or C-terminus. The terms “first” and “second” only serve to correctly and consistently refer to the two different antigen-binding regions in the claims and the description.

Sequence listing:

| Name | Amino acid sequence | SEQ ID NO: |
|---|---|---|
| 5D3 CDR1 | SYAMG | 1 |
| 5D3 CDR2 | AISWSGGTTYYADSVKG | 2 |
| 5D3 CDR3 | SLDCSGPGCHTAEYDY | 3 |
| 6H4 CDR1 | NYGMG | 4 |
| 6H4 CDR2 | GISWSGGSTDYADSVKG | 5 |
| 6H4 CDR3 | VFSGAETAYYPSDDYDY | 6 |
| 6H3 CDR1 | NYGMG | 7 |
| 6H3 CDR2 | GITWSGGSTHYADLVKG | 8 |
| 6H3 CDR3 | VFSGAETAYYPSTEYDY | 9 |
| 6G3 CDR1 | NYGMG | 10 |
| 6G3 CDR2 | GISWSGGSTYYADSVKG | 11 |
| 6G3 CDR3 | VFSGAETAQYPSYDYDY | 12 |
| 5C8 CDR1 | NYAMX₁, wherein X₁ is G or S | 13 |
| 5C8 CDR2 | AISWSGGSTSYADSVKG | 14 |
| 5C8 CDR3 | QFSGADX₂GFGRLGIRGYEYDY, wherein X₂ is Y, F or | 15 |
| | S | |
| 5F5 CDR1 | NYAMG | 16 |
| 5F5 CDR2 | AISWSGGSTYYADSVKG | 17 |
| 5F5 CDR3 | MFSGSESQLVVVITNLYEYDY | 18 |
| 6A1 CDR1 | NYAMG | 19 |
| 6A1 CDR2 | TISWSGGSTYYADSVKG | 20 |
| 6A1 CDR3 | AFSGSDYANTKKEVEYDY | 21 |
| 6E4 CDR1 | DYCIA | 22 |
| 6E4 CDR2 | CITTSDGSTYYADSVKG | 23 |
| 6E4 CDR3 | YFGYGCYGGAQDYRAMDY | 24 |
| 5D3 VHH | EVQLVESGGGLVQAGGSLRLSCAASGRPFSNYAMGWF | 25 |
| | RQAPGKEREFVTVISWSGGSTYYADSVKGRFTISRDNA | |
| | KNTVYLQMNSLKPEDTAVYYCAAQFSGASTVVAGTALD | |
| | YDYWGQGTRVTVSS | |
| 6H4 VHH | EVQLVESGGGLVQAGGSLRLSCAASGRPFSNYGMGWF | 26 |
| | RQAPGKKREFVAGISWSGGSTDYADSVKGRFTISRDN | |
| | AKNTVYLQMNSLKPEDTAVYYCAAVFSGAETAYYPSDD | |
| | YDYWGQGTQVTVSS | |
| 6E3 VHH | EVQLVESGGGLVQAGGSLRLSCAASGRPFSNYGMGWF | 27 |
| | RQAPGKKREFVAGISWSGGSTDYADSVKGRLTISRDN | |
| | AKNTVYLQMNSLKPEDTAVYYCAAVFSGAETAYYPSDD | |
| | YDYWGQGTQVTVSS | |
| 6C1 VHH | EVQLVESGGGLVQAGGSLRLSCAASGRPFSNYGMGWF | 28 |
| | RQAPGKKRESVAGISWSGGSTDYADSVKGRFTISRDN | |
| | AKNTVYLQMNSLKPEDTAVYYCAAVFSGAETAYYPSDD | |
| | YDYWGQGTQVTVSS | |
| 6H3 VHH | EVQLVESGGGLVQAGGSLRLSCAVSGRPFSNYGMGWF | 29 |
| | RQAPGKEREFVAGITWSGGSTHYADLVKGRFTISRDNA | |
| | KNTVHLQMNSLKPEDTAVYYCAAVFSGAETAYYPSTEY | |
| | DYWGQGTQVTVSS | |
| 6G3 VHH | EVQLVESGGGLVQAGGSLRLSCAASGRPFNNYGMGWF | 30 |
| | RQAPGKEREFVAGISWSGGSTYYADSVKGRFTISRDN | |
| | AKNTVYLQMNSLKPEDTAVYYCAAVFSGAETAQYPSYD | |
| | YDYWGQGTQVTVSS | |
| 5C8 VHH var | EVQLVESGGGLVQAGGSLRLSCAASGRPFSNYAM | 31 |
| | X₁WFRQAPGKEREFVAAISWSGGSTSYADSVKGRFTIS | |
| | RDNAKNTVYLQMNSPKPEDTAIYYCAAQFSGAD | |
| | X₂GFGRLGIRGYEYDYWGQGTQVTVSS, wherein X₁ is | |
| | G or S and wherein X₂ is Y, F or S | |
| 5F5 VHH | EVQLVESGGGLVQAGGSLRLSCAASGRTFSNYAMGWF | 32 |
| | RQAPGKEREFVAAISWSGGSTYYADSVKGRFTISRDNA | |
| | KNTVYLQMNSLKPEDTAVYYCAAMFSGSESQLVVVITN | |
| | LYEYDYWGQGTQVTVSS | |
| 6A1 VHH | EVQLVESGGGLVQAGGSLRLSCAASGRTFSNYAMGWF | 33 |
| | RQAPGKEREFVATISWSGGSTYYADSVKGRFTISRDNA | |
| | KNTVYLQMNSLKPEDTAVYYCAAAFSGSDYANTKKEVE | |
| | YDYWGQGTQVTVSS | |
| 6E4 VHH | EVQLVESGGGLVQAGGSLRLSCAASGFTFDDYCIAWFR | 34 |
| | QAPGKEREPVSCITTSDGSTYYADSVKGRFTISSDNAK | |
| | NTVYLQMNRLKPEDTAVYYCAAYFGYGCYGGAQDYRA | |
| | MDYWGKGTLVTVSS | |
| 1D12 CDR1 | DNVMG | 35 |
| 1D12 CDR2 | TIRTGGSTNYADSVKG | 36 |
| 1D12 CDR3 | TIPVPSTPYDY | 37 |
| 1D12 | QVQLVESGGGLVQAGGSLRLSCAASGSMFSDNVMGW | 38 |
| | YRQAPGKQRELVATIRTGGSTNYADSVKGRFTISRDNA | |
| | KNTVYLQMNSLKPEDTAVYYCRHTIPVPSTPYDYWGQG | |
| | TQVTVSS | |
| 5C8 var - | EVQLVESGGGLVQAGGSLRLSCAASGSMFSDNVMGW | 39 |
| 1D12 | YRQAPGKQRELVATIRTGGSTNYADSVKGRFTISRDNA | |
| | KNTVYLQMNSLKPEDTAVYYCRHTIPVPSTPYDYWGQG | |
| | TQVTVSSGGGGSEVQLLESGGGSVQPGGSLRLSCAAS | |
| | GRPFSNYAMSWFRQAPGKEREFVSAISWSGGSTSYAD | |
| | SVKGRFTISRDNSKNTLYLQMNSLRAEDTAVYYCAAQF | |
| | SGADYGFGRLGIRGYEYDYWGQGTQVTVSS | |
| 7D12 CDR1 | SYGMG | 40 |
| 7D12 CDR2 | GISWRGDSTGYADSVKG | 41 |
| 7D12 CDR3 | AAGSAWYGTLYEYDY | 42 |
| 7D12 | EVQLVESGGGSVQTGGSLRLTCAASGRTSRSYGMGWF | 43 |
| | RQAPGKEREFVSGISWRGDSTGYADSVKGRFTISRDN | |
| | AKNTVDLQMNSLKPEDTAIYYCAAAAGSAWYGTLYEYD | |
| | YWGQGTQVTVSS | |
| v19 CDR1 | RSAMG | 44 |
| v19 CDR2 | AIGTRGGSTKYADSVKG | 45 |
| v19 CDR3 | RGPGYPSAAIFQDEYHY | 46 |
| v19 | QVQLQESGGGLVQAGGSLRLSCAASGRTFGRSAMGW | 47 |
| | FRQAPGKEREFVAAIGTRGGSTKYADSVKGRFTISTDN | |
| | ASNTVYLQMDSLKPEDTAVYRCAVRGPGYPSAAIFQDE | |
| | YHYWGQGTQVTVSS | |
| LV1050 | RFMISEYSMH | 48 |
| CDR1 | | |
| LV1050 | TINPAGTTDYADSVKG | 49 |
| CDR2 | | |
| LV1050 | DGYGY | 50 |
| CDR3 | | |
| LV1050 | EVQLVESGGGLVQPGGSLRLSCAASRFMISEYSMHWV | 51 |
| | RQAPGKGLEWVSTINPAGTTDYADSVKGRFTISRDNAK | |
| | NTLYLQMNSLRAEDTAVYYCDGYGYRGQGTQVTVSS | |
| 1D2 CDR1 | GRTASSYVMX₁, wherein X₁ is G or S | 52 |
| 1D2 CDR2 | VINWNGDSTYYX₂DSVKG, wherein X₂ is A or T | 53 |
| 1D2 CDR3 | DTRREWYRDGX₃WGPPARYEYDY, wherein X₃ is Y or | 54 |
| | F | |
| 1D2 | EVQLVESGGGLVQAGGSLRLSCAASGRTASSYVMGWF | 55 |
| | RQAPGKEREFVAVINWNGDSTYYTDSVKGRFAISRDN | |
| | AKNTVYLQMNSLKPEDTAVYYCAADTRREWYRDGYWG | |
| | PPARYEYDYRGQGTQVTVSS | |

### Further Aspects and Embodiments of the Invention

As described above, in a first aspect, the invention relates to a multispecific antibody comprising a first antigen-binding region that binds a human Vγ9Vδ2 T cell receptor and a second antigen-binding region that binds a target expressed on tumor cells for use in the treatment of cancer in a human subject, wherein said human subject is also being treated with, or has also been treated with an aminobisphosphonate (NBP) or other inhibitor of the mevalonate pathway, wherein the inhibitor inhibits farnesyl pyrophosphate synthase or an enzyme further downstream in said pathway, and wherein the first, or a subsequent, administration of said multispecific antibody is performed after the human subject has received two or more administrations of said NBP or other inhibitor.

Thus, similarly, the invention relates to a multispecific antibody comprising a first antigen-binding region that binds a human Vγ9Vδ2 T cell receptor and a second antigen-binding region that binds a target expressed on tumor cells for use in the treatment of cancer in a human subject, wherein said human subject has received two or more administrations of an NBP or of such other inhibitor of the mevalonate pathway.

Thus, similarly, the invention relates to a multispecific antibody comprising a first antigen-binding region that binds a human Vγ9Vδ2 T cell receptor and a second antigen-binding region that binds a target expressed on tumor cells for use in the treatment of cancer in a human subject, wherein said human subject has received two or more administrations of an NBP or of such other inhibitor of the mevalonate pathway prior to the first, or a subsequent, administration of said multispecific antibody.

Thus, similarly, the invention relates to a multispecific antibody comprising a first antigen-binding region that binds a human Vγ9Vδ2 T cell receptor and a second antigen-binding region that binds a target expressed on tumor cells for use in the treatment of cancer in a human subject in combination with an NBP or such other inhibitor of the mevalonate pathway, wherein said human subject receives two or more administrations of said NBP or other inhibitor of the mevalonate pathway prior to the first, or a subsequent, administration of said multispecific antibody.

Similarly, the invention relates to a method for the treatment of cancer, comprising administration of a multispecific antibody comprising a first antigen-binding region that binds a human Vγ9Vδ2 T cell receptor and a second antigen-binding region that binds a target expressed on tumor cells to a human subject, wherein said human subject is also being treated with, or has also been treated with an aminobisphosphonate (NBP) or other inhibitor of the mevalonate pathway, wherein the inhibitor inhibits Farnesyl PP synthase or an enzyme further downstream in said pathway, and wherein the first, or a subsequent, administration of said multispecific antibody is performed after the human subject has received two or more administrations of said NBP or other inhibitor.

Similarly, the invention relates to a method for the treatment of cancer, comprising administration of a multispecific antibody comprising a first antigen-binding region that binds a human Vγ9Vδ2 T cell receptor and a second antigen-binding region that binds a target expressed on tumor cells to a human subject, said method comprising the steps of:
(i) selecting a human subject that is being treated with, or has been treated with, an NBP or other inhibitor of the mevalonate pathway, wherein the inhibitor inhibits Farnesyl PP synthase or an enzyme further downstream in said pathway, wherein the subject has received two or more administrations of said NBP or other inhibitor, and
(ii) administering said multispecific antibody.

Accordingly, the human subject belongs to a subpopulation of the subjects that are eligible for treatment with said multispecific antibody, said subpopulation comprising subjects that are undergoing treatment or have undergone treatment with an NBP or such other inhibitor.

As explained above, it has surprisingly been found that Vγ9Vδ2 T cell-containing PBMCs of patients that have received repetitive treatment with an NBP are still responsive to γδ TCEs and that γδ TCEs can mediate killing of tumor cells by PBMCs of such patients. This allows for cancer treatment regimens wherein γδ TCEs are administered to patients that simultaneously also receive, or have received, repetitive NBP treatment.

Thus, γδ TCE treatment can be given after repetitive treatment with an NBP (or functionally similar inhibitor) or treatment with a γδ TCE and treatment with a NBP (or other inhibitor) can be performed simultaneously. The term “repetitive” when used herein, means more than once, i.e. two or more times.

In one embodiment, the first, or a subsequent, administration of said multispecific antibody is performed after the human subject has received three or more, such as four or more, for example five or more, such as six or more, seven or more, eight or more, nine or more or ten or more administrations of said NBP or other inhibitor.

In another embodiment, the first, or a subsequent, administration of said multispecific antibody is performed within two years, such within one year, e.g. within six months, such as within four months, e.g. within three months, such as within two months, e.g. within one month, of an administration of said NBP or other inhibitor.

In another embodiment, the first administration of said multispecific antibody is performed after the human subject has received two or more administrations of said NBP or other inhibitor.

Some NBPs, such as pamidronate or zoledronate, are typically administered intravenously. Thus, in one embodiment, said administrations of said NBP or other inhibitor are intravenous administrations. In another embodiment, said administrations of said NBP or other inhibitor are not oral administrations.

Dosing frequencies of NBPs typically vary according to compound and mode of administration. For example, an NBP, such as pamidronate may be administered once every three weeks or once every four weeks. Zoledronate may, for example, be administered once every 1, 2 or 3 months or once every 6-12 months.

Thus, in one embodiment, the first, or a subsequent, administration of said multispecific antibody is performed after the human subject has received two or more administrations of said NBP or other inhibitor that were between 2 and 16 weeks apart, for example between 2 and 12 weeks, such as between 3 and 5 weeks, for example 3 or 4 weeks apart.

In one embodiment, the first administration of the multispecific antibody takes place after the human subject has received two or more administrations of an NBP of 3 or 4 weeks apart (i.e. with a time interval of 3 or 4 weeks between the NBP administrations), with the first administration of the multispecific antibody taking place within 6 months, e.g. within 3 or 2 months or within 1 month after administration of an NBP.

In another embodiment, the first, or a subsequent, administration of said multispecific antibody is performed after the human subject has received two or more administrations of said NBP or other inhibitor that were between 4 and 14 months apart, for example from 6 to 12 months apart.

In another embodiment, the first administration of the multispecific antibody takes place after the human subject has received two or more administrations of an NBP of 6 or 12 months apart, with the first administration of the multispecific antibody taking place within 6 months, e.g. within 3 or 2 months or within 1 month after administration of an NBP.

Several NBPs have been described for medical treatment. In one embodiment of the use of the invention, the human subject is treated with an NBP selected from: clodronate, etidronate, pamidronate, alendronate, ibandronate, zoledronate and risedronate. In a further embodiment, the NBP is pamidronate or zoledronate.

In one embodiment, the NBP is pamidronate and the first, or a subsequent, administration of said multispecific antibody is performed after the human subject has received two or more administrations of pamidronate, wherein at least two, or all, of the pamidronate administrations were between 2 and 6 weeks apart, such as between 3 and 5 weeks apart, for example 3 or 4 weeks apart.

In one embodiment, the first administration of the multispecific antibody takes place after the human subject has received two or more administrations of pamidronate of 3 or 4 weeks apart, with the first administration of the multispecific antibody taking place within 6 months, e.g. within 3 or 2 months or within 1 month after an administration of pamidronate. In one embodiment, pamidronate is administered at a dose of 30 mg or 90 mg.

In another embodiment, the NBP is zoledronate and the first, or a subsequent, administration of said multispecific antibody is performed after the human subject has received two or more administrations of zoledronate, wherein at least two, or all, of the zoledronate administrations were between 2 and 16 weeks apart, for example between 1 and 3 months apart, such as 1, 2 or 3 months apart, or 4, 8 or 12 weeks apart.

In one embodiment, the first administration of the multispecific antibody takes place after the human subject has received two or more administrations of zoledronate of 1, 2 or 3 months apart, with the first administration of the multispecific antibody taking place within 6 months, e.g. within 3 or 2 months or within 1 month after an administration of zoledronate. In one embodiment, zoledronate is administered at a dose of 4 mg.

In another embodiment, the NBP is zoledronate and the first, or a subsequent, administration of said multispecific antibody is performed after the human subject has received two or more administrations of zoledronate, wherein at least two, or all, of the zoledronate administrations were between 4 and 14 months apart, for example from 6 to 12 months apart.

In further embodiments of the above described uses according to the invention, the treatment with NBP or other inhibitor continues after administration of the multispecific antibody. Thus, after initiating the treatment with the multispecific antibody, the NBP or other inhibitor is administered at least once more, such as more than 2, 3, 4, 5, 6 or more times after administration of the multispecific antibody. The NBP and antibody treatments thus become simultaneous.

In one embodiment, the human subject is undergoing or, has been enrolled for, a NBP or other inhibitor treatment of a duration of at least 6 months, such as at least 9 months, e.g. 12 months.

Furthermore, the multispecific antibody may be administered as often as required. In one embodiment, the treatment comprises administration of the multispecific antibody more than once, such as between 2 and 100 times, e.g. between 2 and 50 times, e.g. between 2 and 25 times, such as between 6 and 25 times, or between 6 and 12 times, for example twice weekly, weekly, biweekly or with an interval of 3 to 4 weeks.

Thus, for example, in one embodiment, the treatments with NBP (or other inhibitor) and the multispecific antibody may be simultaneous. For example, a patient who has received two or more doses of NBP, with an interval of 3-4 weeks, initiates a treatment with multispecific antibody of a certain duration, e.g. 3-12 months, while continuing 3-4 weekly treatment with NBP. Similarly, a patient who has received treatment with a multispecific antibody may initiate a 3-4 weekly treatment with an NBP, while continuing treatment with the multispecific antibody.

As mentioned above, in some embodiments, the patient is not (only) treated with an NBP, but (also) with another inhibitor of the mevalonate pathway, wherein the inhibitor inhibits farnesyl pyrophosphate synthase (FPPS) or an enzyme further downstream in the mevalonate pathway. In one embodiment, treatment with such other inhibitor leads to the accumulation of isopentenyl pyrophosphate (IPP).

In one embodiment, the inhibitor is an inhibitor of FPPS (UniProtKB-P14324 (FPPS_HUMAN)). In one embodiment, the inhibitor is carnosic acid (Han et al. J. Med. Chem. 2019, 62, 23, 10867-10896). In another embodiment, the inhibitor is an alkylamine, such as iso-butylamine or sec-butylamine (Thompson et al. Blood, 107:651, 2006) In another embodiment, the inhibitor is an inhibitor of geranyl diphospate synthase. In another embodiment, the inhibitor is an inhibitor of isopentemyl isomerase (Wang H et al.] Immunol 2011; 187:5099-5113). In another embodiment, a combination of inhibitors is used, for example a combination of inhibitors of squalene synthase, geranylgeranyl PP synthase and farnesyltransferase.

### Clinical Indications

NBPs are frequently used in the treatment or prevention of osteoporosis, hypercalcemia and osteolytic destruction by metastases of solid tumors (e.g. breast and prostate cancer) or hematological neoplasias (e.g. multiple myeloma).

The use of bisphosphonates, including aminobisphosphonates, in prostate cancer and breast cancer patients has been review by Coleman (2020) Bone 140:115570 and by D'Oronzo et al. (2021) Bone 147:115907, while Mhaskar et al. (2017) Cochrane Database Syst Rev 12 (12) CD003188 provides a review of their use in multiple myeloma (all incorporated herein by reference).

In one embodiment of the use of the invention, the human subject to be treated has been diagnosed with breast cancer, prostate cancer, glioblastoma, lung cancer, renal cancer or multiple myeloma.

The use of NBPs in the treatment or prevention of osteoporosis has been reviewed, e.g. in Grey and Ried Ther Clin Risk Manag. 2006 March; 2 (1): 77-86 and as well in as review by Drake et al. (2008) Mayo Clin Proc. 2008 September; 83 (9): 1032-1045, which also reviews the use of NBPs in Paget disease of bone, malignancies metastatic to bone, multiple myeloma, and hypercalcemia of malignancy.

In one embodiment of the use of the invention, the human subject has been diagnosed with osteoporosis, Paget disease of bone or hypercalcemia, such as hypercalcemia of malignancy.

In some embodiments, the human subject is a subject who is undergoing treatment with an NBP or other inhibitor for treatment or prevention of osteoporosis, Paget disease of bone or hypercalcemia, such as hypercalcemia of malignancy and who is subsequently diagnosed with a (further) cancer disease and treatment for said (further) cancer disease with a multispecific antibody as described herein.

### Multispecific Antibodies

As described above, the invention relates to uses of a multispecific antibody comprising a first antigen-binding region that binds a human Vγ9Vδ2 T cell receptor and a second antigen-binding region that binds a target expressed on tumor cells.

In one embodiment, the multispecific antibody is a bispecific antibody. In one embodiment the multispecific antibody binds the Vδ2 chain of the human Vγ9Vδ2 T cell receptor. In one embodiment the multispecific antibody binds the Vγ9 chain of the human Vγ9Vδ2 T cell receptor.

In one embodiment, the first antigen-binding region is a single-domain antibody and/or the second antigen-binding region is a single-domain antibody.

In one embodiment, the first antigen-binding region is a single-domain antibody selected from the group of antibodies described in WO2015156673 (incorporated herein by reference).

In one embodiment, the first antigen-binding region is a single-domain antibody and comprises the CDR1, CDR2 and CDR3 of:
a) SEQ ID NOs: 1, 2 and 3, respectively;
b) SEQ ID NOs: 4, 5 and 6, respectively;
c) SEQ ID NOs: 7, 8 and 9, respectively;
d) SEQ ID NOs: 10, 11 and 12, respectively
e) SEQ ID NOs: 13, 14 and 15, respectively;
f) SEQ ID NOs: 16, 17 and 18, respectively;
g) SEQ ID NOs: 19, 20 and 21, respectively; or
h) SEQ ID NOs: 22, 23 and 24, respectively.

In one embodiment, the first antigen-binding region comprises a sequence selected from SEQ ID NO:25-34. In another embodiment, the first antigen-binding region comprises a variant of a sequence selected from SEQ ID NO:25-XX, such a variant comprising a sequence having at least 90%, such as least 92%, e.g. at least 94%, such as at least 96%, e.g. at least 98% sequence identity to a sequence selected from the group consisting of SEQ ID NO:25-XX.

In one embodiment, the target expressed on tumor cells is selected from the group consisting of: CD1d, EGFR, CD40, PSMA and CD123.

The term “CD1d”, when used herein, refers to the human CD1d protein (UniProtKB-P15813 (CD1D_HUMAN)).

The term “EGFR”, when used herein, refers to the human EGFR protein (UniProtKB-P00533 (EGFR_HUMAN)).

The term “CD40”, when used herein, refers to the CD40 protein, also known as tumor necrosis factor receptor superfamily member 5 (UniProtKB-P25942 (TNR5_HUMAN)), Isoform I.

The term “PSMA”, when used herein, refers to the human Prostate-Specific Membrane Antigen protein (UniProtKB-Q04609 (FOLH1_HUMAN)).

The term “CD123”, when used herein, refers to the human CD123 protein, also interleukin-3 receptor alpha chain (NCBI Reference Sequence: termed NP_002174.1).

In a further embodiment, the second antigen-binding region is a single-domain antibody and the target expressed on tumor cells is selected from the group consisting of: CD1d, PSMA, EGFR, CD40 and CD123.

In one embodiment, the target expressed on tumor cells is CD1d, i.e. the second antigen-binding region binds CD1d and the cancer to be treated with the antibody is selected from hematological malignancies such as T cell lymphoma, multiple myeloma, acute myeloid leukemia, acute lymphoblastic leukemia, chronic lymphocytic leukemia, mantle cell lymphoma, B cell lymphoma, smoldering myeloma, Hodgkin lymphoma, myelomonocytic leukemias, lymphoplasmacytic lymphoma, hairy cell leukemia, and splenic marginal zone lymphoma, or solid tumors, such as renal cell carcinoma, melanoma, colorectal carcinoma, head and neck cancer, breast cancer, prostate cancer, lung cancer, pancreatic cancer, gastro-esophageal cancer, small bowel carcinoma, central nervous system tumors, medulloblastomas, hepatocellular carcinoma, ovarian cancer, glioma, neuroblastoma, urothelial carcinomas, bladder cancer, sarcoma, penile cancer, basal cell carcinoma, merkel cell carcinoma, neuroendocrine carcinoma, neuroendocrine tumors, carcinoma of unknown primary (CUP), thymoma, vulvar cancer, cervical carcinoma, testicular cancer, cholangiocarcinoma, appendicular carcinoma, mesothelioma, ampullary carcinoma, anal cancer, and choriocarcinoma.

In one embodiment, the second antigen-binding region is a single-domain antibody that binds human CD1d and comprises the CDR1, CDR2 and CDR3 of SEQ ID NOs: 35, 36, and 37, respectively. Such single domain antibodies have been described in WO2016122320 and WO2020060405 (both incorporated by reference). In one embodiment, the antibody comprises the sequence set forth in SEQ ID NO:38. In another embodiment, the second antigen-binding region comprises a variant of SEQ ID NO:38, such a variant comprising a sequence having at least 90%, such as least 92%, e.g. at least 94%, such as at least 96%, e.g. at least 98% sequence identity to the sequence set forth in SEQ ID NO:38. In one embodiment, the antibody comprises the sequence set forth in SEQ ID NO:39. In another embodiment, the antibody comprises a variant of SEQ ID NO:39, such a variant comprising a sequence having at least 90%, such as least 92%, e.g. at least 94%, such as at least 96%, e.g. at least 98% sequence identity to the sequence set forth in SEQ ID NO:39.

In one embodiment, the target expressed on tumor cells is EGFR, i.e. the second antigen-binding region binds EGFR. In one embodiment, the second antigen-binding region is a single-domain antibody that binds human EGFR and comprises the CDR1, CDR2 and CDR3 of SEQ ID NOs: 40, 41, and 42, respectively. Such single domain antibodies have been described in WO2021052995 (incorporated by reference). In one embodiment, the antibody comprises the sequence set forth in SEQ ID NO:43. In another embodiment, the second antigen-binding region comprises a variant of SEQ ID NO:43, such a variant comprising a sequence having at least 90%, such as least 92%, e.g. at least 94%, such as at least 96%, e.g. at least 98% sequence identity to the sequence set forth in SEQ ID NO: 43.

In one embodiment, the target expressed on tumor cells is CD40, i.e. the second antigen-binding region binds CD40 and the cancer to be treated with the antibody is chronic lymphocytic leukemia, multiple myeloma, non-Hodgkin's lymphoma, Hodgkin's lymphoma, follicular lymphoma, head and neck cancer, pancreatic cancer, ovarian cancer, lung cancer, breast cancer, colon cancer, prostate cancer, B-cell lymphoma/leukemia, Burkitt lymphoma or B acute lymphoblastic leukemia.

In one embodiment, the second antigen-binding region is a single-domain antibody that binds human CD40 and comprises the CDR1, CDR2 and CDR3 of SEQ ID NOs: 44, 45, and 46, respectively. Such single domain antibodies have been described in WO2020159368 (incorporated by reference). In one embodiment, the antibody comprises the sequence set forth in SEQ ID NO:47. In another embodiment, the second antigen-binding region comprises a variant of SEQ ID NO: 47, such a variant comprising a sequence having at least 90%, such as least 92%, e.g. at least 94%, such as at least 96%, e.g. at least 98% sequence identity to the sequence set forth in SEQ ID NO:47.

In one embodiment, the target expressed on tumor cells is PSMA, i.e. the second antigen-binding region binds PSMA and the cancer to be treated with the antibody is prostate cancer, colorectal cancer, lung cancer, breast cancer, endometrial and ovarian cancer, gastric cancer, renal cell cancer, urothelial cancer, hepatocellular cancer, oral squamous cancer, thyroid tumors or glioblastoma.

In one embodiment, the second antigen-binding region is a single-domain antibody that binds human PSMA and comprises the CDR1, CDR2 and CDR3 of SEQ ID NOs: 48, 49, and 50, respectively. In one embodiment, the antibody comprises the sequence set forth in SEQ ID NO:51. In another embodiment, the second antigen-binding region comprises a variant of SEQ ID NO:51, such a variant comprising a sequence having at least 90%, such as least 92%, e.g. at least 94%, such as at least 96%, e.g. at least 98% sequence identity to the sequence set forth in SEQ ID NO:51.

In one embodiment, the target expressed on tumor cells is CD123, i.e. the second antigen-binding region binds CD123 and the cancer to be treated with the antibody is for use in the treatment of acute myeloid leukemia, B-cell acute lymphoblastic leukemia, hairy cell leukemia, Hodgkin lymphoma, blastic plasmacytoid dendritic neoplasm, chronic myeloid leukemia, chronic lymphocytic leukemia, B-cell chronic lymphoproliferative disorders or myelodysplastic syndrome.

In one embodiment, the second antigen-binding region is a single-domain antibody that binds human CD123 and comprises the CDR1, CDR2 and CDR3 of SEQ ID NOs: 52, 53, and 54, respectively. In one embodiment, the antibody comprises the sequence set forth in SEQ ID NO: 55. In another embodiment, the second antigen-binding region comprises a variant of SEQ ID NO: 55, such a variant comprising a sequence having at least 90%, such as least 92%, e.g. at least 94%, such as at least 96%, e.g. at least 98% sequence identity to the sequence set forth in SEQ ID NO:55.

The multispecific antibody used in the invention may be of any of the multispecific, such as bispecific formats, described herein and may be human, humanized or chimeric or a combination of these, e.g. having one antigen-binding region humanized and the other not.

The multispecific antibody used in the invention may or may not comprise constant region sequences, such as an Fc region. The constant regions of an antibody, if present, may mediate the binding of the immunoglobulin to host tissues or factors, including various cells of the immune system (such as effector cells and T cells) and components of the complement system such as C1q, the first component in the classical pathway of complement activation. In some embodiments, however, the Fc region of the antibody has been modified to become inert, “inert” means an Fc region which is at least not able to bind any Fcγ Receptors, induce Fc-mediated cross-linking of FcRs, or induce FcR-mediated cross-linking of target antigens via two Fc regions of individual antibodies. In a further embodiment, the inert Fc region is in addition not able to bind C1q. In one embodiment, the antibody contains mutations at positions 234 and 235 (Canfield and Morrison (1991) J Exp Med 173:1483), e.g. a Leu to Phe mutation at position 234 and a Leu to Glu mutation at position 235 (according to the EU-numbering, see below). In another embodiment, the antibody contains a Leu to Ala mutation at position 234, a Leu to Ala mutation at position 235 and a Pro to Gly mutation at position 329. In another embodiment, the antibody contains a Leu to Phe mutation at position 234, a Leu to Glu mutation at position 235 and an Asp to Ala at position 265.

All references, articles, publications, patents, patent publications, and patent applications cited herein are incorporated by reference in their entireties for all purposes. However, mention of any reference, article, publication, patent, patent publication, and patent application herein is not, and should not be, taken as acknowledgment or any form of suggestion that they constitute valid prior art or form part of the common general knowledge in any country in the world.

### EXAMPLES

Peripheral blood mononuclear cells (PBMCs) were isolated from seven patients diagnosed with metastatic breast cancer that had been treated with an NBP for a period ranging from 3 months to 2.5 years. From these patients, three had been treated with pamidronate, three with zoledronate, and one patient was treated with oral clodronic acid. Furthermore, PMBCs were isolated from a glioblastoma multiforme patient that had been treatment with monthly doses of zoledronate for one year. From 6 subjects, PMBCs were isolated between 2 and 12 weeks after the last NBP administration.

The antibody used was a bispecific γδ TCE, binding the Vδ2 chain of the Vγ9Vδ2 T cell receptor and tumor target CD1d. The antibody has been described in SEQ ID NO: 87 of WO2020060405.

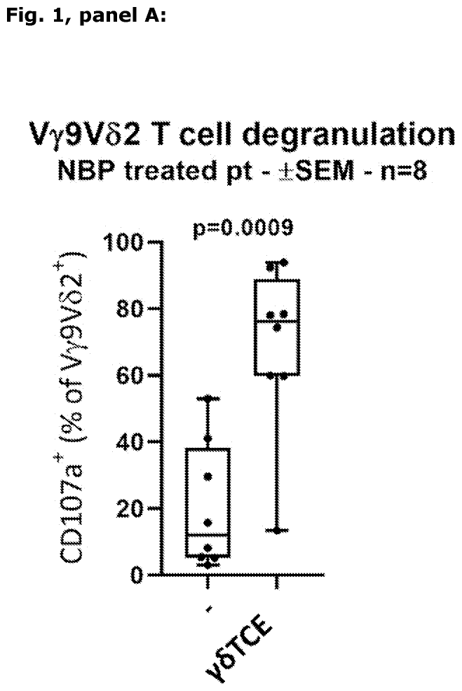

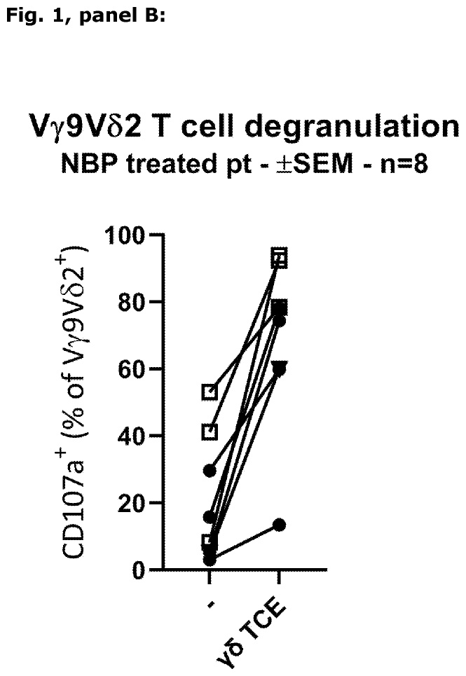

10\hat{ }5 PBMCs were cocultured with 2.5*10\hat{ }4 MM1s.CD1d target cells (Lameris, R., et al (2020) Nat Cancer 1, 1054-1065), in the presence of the targeting γδ TCE or medium control. Following overnight culture, CD107a exposure on Vγ9Vδ2 T cells was assessed using flow cytometry as a measure for degranulation. Coculturing PBMCs with the target cell line induced a mean CD107a expression of 20.2% on Vγ9Vδ2 T cells, which significantly increased to 68.8% in the presence of γδ TCE (p=0.0009, n=8) (FIG. 1A). For each individual patient, CD107a expression increased in the presence of γδ TCE (FIG. 1B).

This demonstrates that Vγ9Vδ2 T cells from such NBP pre-treated patients were responsive to activation by γδ TCEs.

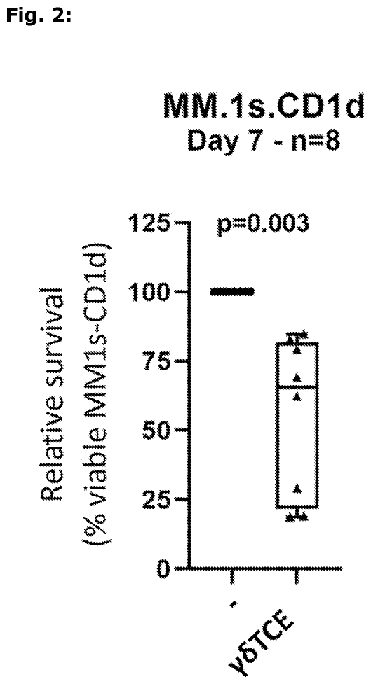

Relative target cell lysis following Vγ9Vδ2 T cell engagement was determined by coculturing 10\hat{ }5 PBMCs with 2.5*10\hat{ }4 MM1s.CD1d target cells for 7 days, in the presence of γδ TCE vs a medium control (−). The absolute number of viable (7AAD-) target cells was quantified using counting beads for flow cytometry. The relative survival of MM1s.CD1d target cells significantly reduced to a mean of 55.6% in the presence of the γδ TCE, as compared to the medium control (p=0.003, n=8) (FIG. 2).

This demonstrates that Vγ9Vδ2 T cell-containing PBMCs from such NBP pre-treated patients were able to kill target tumor cells in the presence γδ TCEs.

PBMCs were cocultured in a 4:1 ratio with MM1s.CD1d target cells, with γδ TCE or a medium control. The absolute number of Vγ9Vδ2 T cells was quantified using counting beads for flow cytometry. Vγ9Vδ2 T cell expansion was assessed at day 7 and 14 and plotted relative to day 0.

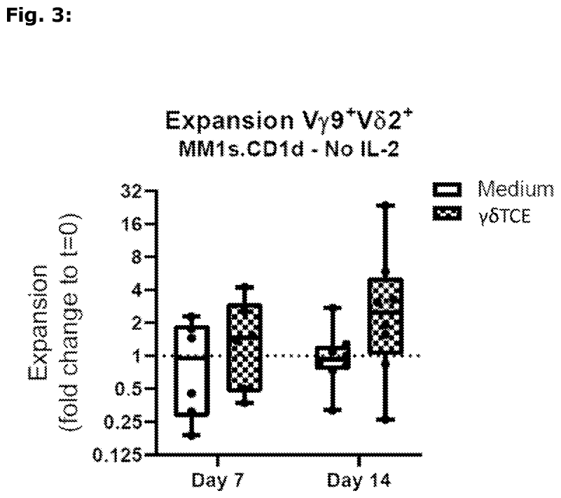

As compared to the medium control, an increase in Vγ9Vδ2 T cell numbers following γδ TCE engagement was observed at day 7 (1.1-vs 1.8-fold) and day 14 (1.1-vs 5.1-fold) (p=ns, n=8) (FIG. 3).

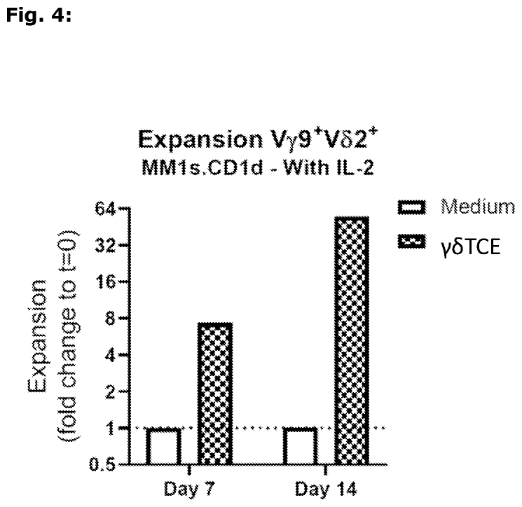

In a similar experiment, PBMCs were cocultured in a 4:1 ratio with MM1s.CD1d target cells, with γδ TCE or a medium control, in the presence of 100 U/ml IL-2. The absolute number of Vγ9Vδ2 T cells was quantified using counting beads for flow cytometry. Vγ9Vδ2 T cell expansion was assessed at day 7 and 14 and plotted relative to day 0. As compared to the medium control, a strong increase in Vγ9Vδ2 T cell numbers following γδ TCE engagement was observed at day 7 (1.0-vs 7.3-fold) and day 14 (1.0-vs 54.7-fold) (n=1) (FIG. 4).

This demonstrates that Vγ9Vδ2 T cells from such NBP pre-treated patients were able expand in the presence γδ TCEs. In these in vitro conditions, the expansion was stronger when IL-2 was added.

1-18. (canceled)

19. A method of treating a cancer in a subject in need thereof comprising administering:
a. a multispecific antibody comprising a first antigen-binding region that binds a human Vγ9Vδ2 T cell receptor and a second antigen-binding region that binds a target expressed on a tumor cell; and
b. an inhibitor of a mevalonate pathway;
wherein the subject is being treated with or has been treated with an inhibitor of the mevalonate pathway.

20. The method of claim 19, wherein the inhibitor of the mevalonate pathway inhibits farnesyl pyrophosphate synthase or an enzyme further downstream in said pathway.

21. The method of claim 20, wherein the inhibitor of the mevalonate pathway is an aminobisphosphonate (NBP).

22. The method of claim 21, wherein the first, or a subsequent, administration of the multispecific antibody is administered after the subject has received two or more administrations of the NBP.

23. The method of claim 22, wherein the first, or a subsequent, administration of the multispecific antibody is administered within two years of an administration of the NBP.

24. The method of claim 23, wherein the NBP is administered intravenously.

25. The method of claim 24, wherein the two or more administrations of the NBP are between 2 and 16 weeks apart.

26. The method of claim 21, wherein the NBP is selected from clodronate, etidronate, pamidronate, alendronate, ibandronate, zoledronate and risedronate.

27. The method of claim 26, wherein the NBP is pamidronate or zoledronate.

28. The method of claim 27, wherein:
a. the NBP is pamidronate and wherein the first, or a subsequent, administration of the multispecific antibody is performed after the human subject has received two or more administrations of pamidronate, wherein at least two, or all, of the pamidronate administrations were between 2 and 6 weeks apart; or
b. the NBP is zoledronate and wherein the first, or a subsequent,
administration of the multispecific antibody is performed after the human subject has received two or more administrations of zoledronate, wherein at least two, or all, of the zoledronate administrations were between 2 week and 12 months apart.

29. The method of claim 19, wherein the treatment with inhibitor continues after administration of the multispecific antibody.

30. The method of claim 29, wherein the treatment comprises administration of the multispecific antibody between 2 and 100 times.

31. The method of claim 19, wherein the human subject has been diagnosed with breast cancer, prostate cancer, glioblastoma, lung cancer, renal cancer or multiple myeloma.

32. The method of claim 19, wherein the human subject has been diagnosed with osteoporosis, Paget disease of bone or hypercalcemia.

33. The method of claim 19, wherein the cancer expresses a target antigen selected from: CD1d, PSMA, EGFR, CD40 and CD123.

34. The method of claim 19, wherein the multispecific antibody is a bispecific antibody.

35. The method of claim 34, wherein the bispecific antibody comprises
a. a first antigen-binding region comprising a complementarity determining region (CDR) 1 sequence selected from SEQ ID NOs: 1, 4, 7, 10, 13, 16, 19, and 22; a CDR2 sequence selected from SEQ ID NOs: 2, 5, 8, 11, 14, 17, 20, and 23; and a CDR3 sequence selected from SEQ ID NOs: 3, 6, 9, 12, 15, 18, 21, and 24; and
b. a second antigen-binding region comprising CDR1 sequence selected from SEQ ID NOs: 35, 40, 44, 48, and 52; a CDR2 sequence selected from SEQ ID NOs: 36, 41, 45, 49, and 53; and a CDR3 sequence selected from SEQ ID NOs: 37, 42, 46, 50, and 54.
